(19) United States
(12) Patent Application Publication
(10) Pub. No.: US 2018/0200476 A1
(43) Pub. Date: Jul. 19, 2018

(54) NON-INVASIVE BRAIN TEMPERATURE REGULATING DEVICES FOR ENHANCING SLEEP

(71) Applicants: Robert E. TUCKER, Sanibel, FL (US); Jeffrey J. SCHIRM, Monroeville, PA (US)

(72) Inventors: Robert E. TUCKER, Sanibel, FL (US); Jeffrey J. SCHIRM, Monroeville, PA (US)

(21) Appl. No.: 15/921,528

(22) Filed: Mar. 14, 2018

Related U.S. Application Data
(63) Continuation-in-part of application No. 14/435,515, filed on Apr. 14, 2015, now abandoned.
Application No. 14/435,515 is a continuation-in-part of application No. 15/597,057, filed on May 16, 2017.
Application No. 15/597,057 is a continuation-in-part of application No. 14/749,590, filed on Jun. 24, 2015, now Pat. No. 9,669,185.
Application No. 14/749,590 is a continuation of application No. 13/868,015, filed on Apr. 22, 2013, now Pat. No. 9,089,400.
Application No. 13/868,015 is a continuation of application No. 13/019,477, filed on Feb. 2, 2011, now Pat. No. 8,425,583.
Application No. 13/019,477 is a continuation-in-part of application No. 11/788,694, filed on Apr. 20, 2007, now Pat. No. 8,236,038.
Application No. 13/019,477 is a continuation-in-part of application No. 12/288,417, filed on Oct. 20, 2008, now Pat. No. 9,492,313.
(60) Provisional application No. 61/727,054, filed on Nov. 15, 2012.
(60) Provisional application No. 61/859,161, filed on Jul. 26, 2013.
(60) Provisional application No. 61/300,768, filed on Feb. 2, 2010.
(60) Provisional application No. 60/793,680, filed on Apr. 20, 2006.

(51) Int. Cl. A61M 21/02; A61M 19/00; A61F 7/00; A61F 7/10; A61M 21/00; A61B 17/00

(52) U.S. Cl. CPC A61M 21/02; A61M 2210/06; A61F 7/0085; A61F 7/10; A61M 21/00; A61M 2205/3606; A61M 2021/0083; A61M 2021/0066; A61F 2007/0095; A61F 2007/0075; A61F 2007/0056; A61F 2007/0007; A61F 2007/0002; A61F 7/007; A61M 2205/3626; A61M 2209/088; A61B 2017/00132; A61M 2210/0693; A61M 19/00

(57) ABSTRACT

Methods, systems and devices for enhancing sleep, including enhancing the quality of sleep, reducing sleep onset time, increasing total sleep time, treating insomnia, and/or treating other neurological disorders by non-invasive temperature regulation of the frontal cortex prior to and/or during sleep. Described herein are thermal applicators that include phase change materials and/or evaporative cooling, as well as headgear for securing the applicators comfortably against the appropriate region of the user's head.

### CROSS REFERENCE TO RELATED APPLICATIONS

This patent claims priority as a continuation-in-part of U.S. patent application Ser. No. 14/435,515, titled “NON-INVASIVE BRAIN TEMPERATURE REGULATING DEVICES FOR ENHANCING SLEEP,” filed on Apr. 14, 2015, now U.S. Patent Application Publication No. 2015/0238725, which is a 371 of International Patent Application No. PCT/US2013/070251, filed Nov. 15, 2013, now International Publication No. WO 2014/078630, which claims priority to U.S. Provisional Patent Application No. 61/727,054, filed Nov. 15, 2012, titled “NON-INVASIVE BRAIN COOLING DEVICES FOR ENHANCING SLEEP” and U.S. Provisional Patent Application No. 61/859,161, filed Jul. 26, 2013, titled “APPARATUS AND METHOD FOR MODULATING SLEEP,” each of which are herein incorporated by reference in their entirety.

This patent also claims priority as a continuation-in-part of U.S. patent application Ser. No. 15/597,057, titled “FOREHEAD COOLING METHOD AND DEVICE TO STIMULATE THE PARASYMPATHETIC NERVOUS SYSTEM FOR THE TREATMENT OF INSOMNIA,” filed on May 16, 2017, now U.S. Patent Application Publication No. 2017/0252534, which claims priority as a continuation-in-part of U.S. patent application Ser. No. 14/749,590, filed on Jun. 24, 2015, titled “METHODS, DEVICES AND SYSTEMS FOR TREATING INSOMNIA BY INDUCING FRONTAL CEREBRAL HYPOTHERMIA,” now U.S. Pat. No. 9,669,185, which claims priority as a continuation of U.S. patent application Ser. No. 13/868,015, filed Apr. 22, 2013, titled “METHODS, DEVICES AND SYSTEMS FOR TREATING INSOMNIA BY INDUCING FRONTAL CEREBRAL HYPOTHERMIA,” now U.S. Pat. No. 9,089,400, which is a continuation of U.S. patent application Ser. No. 13/019,477, filed Feb. 2, 2011, titled “METHODS, DEVICES AND SYSTEMS FOR TREATING INSOMNIA BY INDUCING FRONTAL CEREBRAL HYPOTHERMIA,” now U.S. Pat. No. 8,425,583, which claims priority to U.S. Provisional Patent Application No. 61/300,768, filed Feb. 2, 2010 and titled “FRONTAL CEREBRAL HYPOTHERMIA AS A TREATMENT FOR INSOMNIA AND NEUROLOGICAL AND PSYCHIATRIC DISORDERS,” each of which are herein incorporated by reference in their entirety.

U.S. patent application Ser. No. 13/019,477 also claims priority as a continuation-in-part of U.S. patent application Ser. No. 11/788,694, filed Apr. 20, 2007, titled “METHOD AND APPARATUS OF NONINVASIVE, REGIONAL BRAIN THERMAL STIMULATION FOR THE TREATMENT OF NEUROLOGICAL DISORDERS,” now U.S. Pat. No. 8,236,038, which claims priority to U.S. Provisional Patent Application No. 60/793,680, filed on Apr. 20, 2006, each of which are herein incorporated by reference in their entirety.

U.S. patent application Ser. No. 13/019,477 also claims priority as a continuation-in-part of U.S. patent application Ser. No. 12/288,417, filed Oct. 20, 2008, titled “METHOD AND APPARATUS OF NONINVASIVE, REGIONAL BRAIN THERMAL STIMULATION FOR THE TREATMENT OF NEUROLOGICAL DISORDERS,” now U.S. Pat. No. 9,492,313, which is herein incorporated by reference in its entirety.

This application may be related to the following patents and pending applications, each of which is herein incorporated by reference in its entirety: U.S. Pat. No. 8,236,038, filed Ser. No. 11/788,694 (titled “METHOD AND APPARATUS OF NONINVASIVE, REGIONAL BRAIN THERMAL STIMULI FOR THE TREATMENT OF NEUROLOGICAL DISORDERS”); U.S. patent application Ser. No. 13/019,477, filed Feb. 2, 2011 (titled “METHODS, DEVICES AND SYSTEMS FOR TREATING INSOMNIA BY INDUCING FRONTAL CEREBRAL HYPOTHERMIA”); and U.S. patent application Ser. No. 12/288,417, filed Oct. 20, 2008 (titled “METHOD AND APPARATUS OF NONINVASIVE, REGIONAL BRAIN THERMAL STIMULI FOR THE TREATMENT OF NERUOLOGICAL DISORDERS”).

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are apparatuses (e.g., device and systems) and methods for enhancing sleep, including in particular enhancing the quality of sleep, reducing sleep onset time, increasing total sleep time, treating insomnia and treating other neurological disorders by non-invasively regulating the temperature of the frontal cortex prior to and/or during sleep.

### BACKGROUND

Sleep is essential for a person's health and wellbeing, yet millions of people do not get enough sleep and many suffer from lack of sleep. Surveys conducted by the U.S. National Science Foundation between 1999 and 2004 found that at least 40 million Americans suffer from over 70 different sleep disorders, and 60 percent of adults report having sleep problems a few nights a week or more. Most of those with these problems go undiagnosed and untreated. In addition, more than 40 percent of adults experience daytime sleepiness severe enough to interfere with their daily activities at least a few days each month, with 20 percent reporting problem sleepiness a few days a week or more. Furthermore, 69 percent of children experience one or more sleep problems a few nights or more during a week.

Insomnia is the most common sleep complaint across all stages of adulthood, and for millions of people, the problem is chronic. Many health and lifestyle factors can contribute to insomnia including stress, depression, medical illnesses, pain, medications, or specific sleeping disorders. There is great need for additional research to better define the nature of chronic insomnia.

Existing treatments of neurological and/or sleeping disorders, including insomnia, include the use of over the counter or prescription drugs and/or behavioral treatments. Prescription drugs are known to aid patients suffering from sleeping disorders, however, these drugs can be quite expensive and potentially addicting. Some medications even become less effective as use continues. Additionally, the prescriptions can have unwanted and harmful side effects.

Other techniques to treat sleeping disorders include a variety of behavioral measures including stimulus control therapy, sleep restriction therapy, relaxation training, cognitive therapy, and sleep hygiene education. While these measures have moderate effectiveness, they are costly, require significant time to implement and require highly trained clinicians to implement.

One treatment technique previously described addresses these issues by using non-invasive and localized or regional thermal stimuli to the brain that helps treat sleep disorders, including insomnia. Specifically, this method may help restore or mimic normal function in the cerebral cortex. The restoration of function in the cerebral cortex plays a significant role in sleep. At the molecular and neuronal levels, hypothesized functions of sleep include the restoration of brain energy metabolism through the replenishment of brain glycogen stores that are depleted during wakefulness and the downscaling of synapses that have been potentiated during waking brain function. A homeostatic sleep drive, or pressure for sleep, is known to build throughout the waking hours and then is discharged during sleep. At the electroencephalographic (EEG) level, this is measured by EEG spectral power in the delta (0.5-4 Hz) frequency band.

These sleep-related processes have some regional specificity for the prefrontal cortex. Slow wave sleep rhythms have both thalamic and cortical components. An anterior dominance of EEG spectral power in the delta EEG spectral power range has been reported. A frontal predominance for the increase in delta power following sleep loss has been also reported. This region of the cortex also plays a prominent role in waking executive functions which are preferentially impaired following sleep deprivation. These sleep deprivation induced cognitive impairments have been related to declines in frontal metabolism after sleep loss. While cerebral metabolism declines globally from waking to NREM sleep, these declines are most pronounced in heteromodal association cortex, including the prefrontal cortex.

Insomnia is associated with global cerebral hypermetabolism. Nofzinger et al. (Am J Psychiatry, 2004) assessed regional cerebral glucose metabolism during both waking and NREM sleep in insomnia patients and healthy subjects using [18F] fluoro-2-deoxy-D-glucose positron emission tomography (PET). Insomnia patients show increased global cerebral glucose metabolism during sleep and wakefulness; and a smaller decline in relative metabolism from wakefulness to sleep in wake-promoting regions of the brain. In a comparison between insomnia and depressed patients, insomnia patients demonstrated increased waking relative metabolism in the prefrontal cortex. Finally, recent research has shown that the amount of wakefulness after sleep onset, or WASO, in insomnia patients correlates with increasing metabolism in the prefrontal cortex during NREM sleep.

The relationship between body temperature and quality of sleep generally has been described in connection with prior research in the field of sleep medicine. Heat loss, via selective vasodilatation of distal skin regions (measured by the distal minus proximal skin temperature gradient (DPG), seems to be a crucial process for the circadian regulation of core body temperature (CBT) and sleepiness (Aschoff 1956; Krauchi and Wirz-Justice 1994, 2002; Krauchi et al. 1998, 2000). Increased DPG before lights off has been noted to promote a rapid onset of sleep, suggesting a link between thermoregulatory and arousal (sleepiness) systems (Krauchi et al. 1999, 2000). Hot environments impair the sleep process including falling asleep and maintaining sleep as well as generating slow wave sleep as the increased ambient temperature interferes with the normal declines in core body temperature associated with the sleep onset process. Finally, rapid and intense temperature drops around the sleep onset or sleeping periods are expected to have an arousing effect (Horne and Reyner 1999; Hayashi et al. 2003). In contrast, the apparatuses and methods described herein minimize such adverse effects from temperature changes through application of a controlled (including relatively constant) thermal regulation over a prolonged period of time to a localized surface of the scalp. Thus, it has been found that noninvasive, regional thermal stimulus to the scalp (e.g., between 10 degrees C. and 40 degrees C.) of the head may help adjust metabolism in the cerebral cortex underlying the stimulus and, thereby, provide treatment for neurological disorders.

Previously described technologies for brain temperature regulation (e.g., cooling) may use a cooling apparatus configured to be placed over the scalp/head immediately atop the frontal cortex region, and cooling of the apparatus is typically applied by circulating coolant, although other cooling mechanisms are discussed. Described herein are advancements and further refinements of this early work, expanding the types of thermal regulation apparatuses that may be used, as well as ways for securing the apparatus to the proper region of a patient's head.

### SUMMARY OF THE DISCLOSURE

In general, described herein are non-invasive methods and apparatuses (including devices and systems) for applying thermal therapy to the skin over the prefrontal cortex. In some variations, the apparatuses and methods of using them to enhance sleep accomplish sustained thermal regulation (warming or cooling) in an appropriate therapeutic range and time using one or more phase change materials. Also described are devices and methods to enhance sleep that accomplish sustained thermal regulation (cooling) in an appropriate therapeutic range and time using sustained evaporative cooling to enhance sleep. Finally, also descried herein is headgear that is specifically adapted to hold a thermal applicator to provide sustained thermal regulation in the appropriate anatomical region of the head.

In many of the therapeutic methods described herein, the apparatuses (devices or systems) include and applicator having a thermal transfer region and a phase change material that is configured to contact or be placed in thermal contact, with the patient's skin; specifically the skin over the prefrontal cortex. The thermal transfer region may be further temperature controlled by any appropriate thermal regulator region, particularly passive thermal regulator regions, which do not require active heating/cooling (by an electrically powered devices such as a heater/chiller, Peltier, etc.). For example, a passive thermal regulator may include a phase change material, evaporative cooling, or some combination thereof. Phase changing materials and sustained evaporative cooling may be used specifically to provide appropriate therapeutic cooling in various embodiments a described herein. Although passive thermal regulators are described in particular detail here, any of the applicators and methods described herein (unless the context indicates otherwise) may include an active thermal regulator in addition or in alternative. An active thermal regulator may include a fluid cooled/warmed, a solid state (e.g., Peltier device), or the like.

Also described herein are methods of enhancing the sleep, such as enhancing the quality of sleep, reducing sleep onset, sustaining sleep and/or treating insomnia by non-invasively applying thermal regulation to the subject's frontal cortex using an applicator including a phase-changing cooling region and/or evaporative cooling region. In general, these methods may include: positioning an applicator having a phase change material or evaporative cooling region so that a thermal transfer region is in communication with the subject's skin over the prefrontal cortex, as shown in FIGS. 1 and 2; and regulating (holding) the temperature at a predetermined temperature (e.g., a temperature between 10 degree C. and 40 degrees C.) for a predetermined time (e.g., 30 min, 1 hr, 2 hrs, 3 hrs, 4 hrs, 5 hrs, 6 hrs, 7 hrs, 8 hrs) using a phase changing material having a transition temperature at about the predetermined temperature, and maintaining the temperature within prescribed limits for the predetermined time period of at least 15 minutes and up to 480 minutes or more.

For example, described herein are applicators to enhance sleep by regulating the temperature of a subject's frontal cortex when worn. An applicator may include: a thermal regulator region comprising a phase change material having a phase transition between about 10 degrees C. and about 40 degrees C.; a thermal transfer region in thermal communication with the thermal regulator region, wherein the thermal transfer regions is configured to conform to and to contact a subject's forehead so that the thermal transfer region is positioned against the subject's head over the frontal cortex; and a strap configured to hold the applicator against the subject's head when the subject is sleeping.

Any appropriate phase change material may be used. For example, a phase change material may be a homogenous material (e.g., all a single material) or the phase change material may be made up of a plurality of different phase change materials, each having a different phase transition temperature. The phase transition temperature in the case of a phase transition material that comprises a mixture of different component phase transition materials may be the temperature (or temperatures) at which the temperature of the thermal regulator sustains by the passive release/absorption of energy during use (and following pre-chilling or pre-warming before use), for example, a temperature between about 10 degrees C. and about 40 degrees C.

In some variations the thermal regulator includes a plurality of smaller bodies including the phase change material. These smaller bodies may be capsules, or may otherwise encapsulate the phase change material. For example the thermal regulator may comprise a plurality of capsules, wherein each capsule encapsulates the phase change material. These capsules may be connected by another material (e.g., the material of the thermal transfer region or a different material), or suspended in another material. The material in which the capsules are held typically has a relatively high thermal conductivity (e.g., greater than about 0.1 watts per meter kelvin (W/(m*K)), 0.2 W/m*K, 0.3 W/m*K, 0.4 W/m*K, 0.5 W/m*K, 0.5 W/m*K, 0.7 W/m*K, 0.8 W/m*K, 0.9 W/m*K, 1 W/m*K, 2 W/m*K, 5 W/m*K, etc.). In some variations the thermal regulator region comprises a plurality of capsules each encapsulating the phase change material, wherein the capsules are arranged in a matrix of thermally conductive and conformable material.

In some variations the thermal regulator is formed of a single body. For example, the thermal regulator may comprise a single body comprising the phase change material.

As mentioned, the phase change material may be any appropriate phase change material having a phase transition temperature at the appropriate temperature. For example, the phase transition material may be an organic phase change material, such as a paraffin. As mentioned, the phase change material may comprise a mixture of two or more different phase change materials.

In general, the thermal regulator may be configured so that the phase change material is sustained at the phase transition temperature for greater than a minimum time (e.g., 15 minutes, 30 minutes, 45 minutes, 1 hour, 2 hours, 3 hours, 4 hours, 5 hours, 6 hours, 7 hours, 8 hours, or 15 minute increments of any of these). The duration that the phase change material is held at its phase transition temperature may depend primarily on the nature of the phase change material, and is related to the rate at which the phase change material releases or absorbs thermal energy as it changes phase. Other factors, including the amount of phase change material, and the ambient (surrounding) temperature and pressure may also effect the duration. However, assuming that the phase change material begins with the material completely at an initial phase (e.g., solid or liquid), and assuming that the ambient temperature (e.g., air temperature and the skin temperature of the subject wearing the applicator) are generally the same when comparing different applicators, the material properties of the phase change material as well as the amount of phase change material may primarily determine the duration that the phase change material is held at its phase transition temperature. Thus, the thermal regulator may be configured so that the phase change material is maintained at about the phase transition temperature for greater than about 30 minutes when the applicator is worn by a subject. In some variations, the thermal regulator is configured so that the phase change material is maintained at about the phase transition temperature for greater than about 6 hour when the applicator is worn by a subject.

In general, the thermal transfer region is configured to transfer thermal energy between the region of the subject's head over the frontal cortex and the thermal regulator region. Thus, the thermal transfer regions may be formed of a material having a relatively high thermal conductivity. The thermal transfer region may also be configured to be flexible and/or form-fitting over the patient's head, to optimize the contact and transfer of thermal energy. For example, the thermal transfer region may include a material having a thermal conductivity of greater than about 0.1 watts per meter kelvin (W/(m*K)) (e.g., greater than about 0.2 W/m*K, 0.3 W/m*K, 0.4 W/m*K, 0.5 W/m*K, 0.5 W/m*K, 0.7 W/m*K, 0.8 W/m*K, 0.9 W/m*K, 1 W/m*K, 2 W/m*K, 5 W/m*K, etc.).

The thermal transfer region may generally be configured to position the thermal regulator over just the subject's frontal cortex when the applicator is worn by the subject, or primarily over just the subject's frontal cortex (e.g., over just the frontal cortex and immediately adjacent regions). For example, the thermal transfer region may be configured to contact the subject's forehead but not to contact the subject's periorbital or cheek regions of the subject's face when the applicator is worn by the subject. The thermal transfer region may be configured to contact the subject's forehead but not to contact the back of the subject's head when the applicator is worn by the subject, and/or the back and sides of the subject's head (generally excluding the temples).

As mentioned, the thermal transfer region may include a layer of thermally conductive material configured to contact the subject's forehead when the applicator is worn by the subject. Examples of thermally conductive materials include fabrics (and/or coated fabrics, etc.) having a relatively high thermal conductivity (e.g., greater than 0.1, 0.2, 0.3, 0.4, 0.5, etc. W/(m*K)). The thermal conductivity may be enhanced by include a high thermal conductivity coating (e.g., diamond-like coatings, metal oxides, nitrides, carbides, glass, etc.)

Any of the applicators described herein may include an attachment to secure the applicator to the subject's head. For example, an applicator may include a strap configured as a headgear. The headgear may contact any portion of the subject's head, including the face, eye orbit, etc.) but typically does not provide thermal contact between the thermal regulatory region and the subject's head. Thus, the exchange of thermal energy between the thermal regulatory region and the subject's head may be relatively limited by the thermal transfer region to the region of the subject's head above the frontal cortex. For example, a headgear may include a headband, hat, cap, kerchief, or the like, for holding the thermal transfer region in contact with the appropriate region of the head (e.g., the forehead/scalp), but preventing thermal contact between the thermal regulatory region and the rest of the head/face.

Any of the applicators to enhance a subject's sleep by regulating the temperature of the frontal cortex when worn may include: a thermal regulator region comprising a plurality of bodies each enclosing a phase change material having a phase transition between about 10 degrees C. and about 40 degrees C.; a thermal transfer region in thermal communication with the thermal regulator region, wherein the thermal transfer regions is configured to conform to and to contact a subject's head so that the thermal transfer region is positioned against the subject's head over the frontal cortex, further wherein the thermal transfer region is configured to contact and the subject's forehead but not to contact the subject's periorbital or cheek regions of the subject's face to regulate temperature when the applicator is worn by the subject; and a strap configured to hold the thermal transfer region against the subject's head when the subject is sleeping.

Also described herein are methods of enhancing sleep. In general, a method of enhancing sleep may include a method of reducing the sleep onset time, and/or prolonging the duration of sleep (e.g., increasing total sleep time), and/or increasing the quality of sleep, and/or, treating insomnia, and/or treating other neurological disorders by non-invasive temperature regulation of the frontal cortex before or after sleep onset.

For example, described herein are methods of enhancing sleep in a subject, the method comprising: positioning an applicator having a thermal regulator region comprising a plurality of bodies each enclosing a phase change material having a phase transition between about 10 degrees C. and about 40 degrees C. and a thermal transfer region in thermal communication with the thermal regulator region so that the thermal transfer region contacts the subject's forehead but does not contact the periorbital or cheek regions of the subject's face; and maintaining the temperature of the thermal transfer region at the phase transition temperature to enhance the subject's sleep.

The step of positioning may comprise positioning the applicator so that the thermal transfer region does not contact the top or back of the subject's head. In any of these variations, the step of positioning comprises adjusting a headgear to hold the applicator to the subject's head.

Maintaining may mean maintaining the temperature of the thermal regulator region at the phase transition temperature for at least some minimum time (e.g., 30 minutes, 60 min, 2 hours, 3 hours, 4 hours, 5 hours, 6 hours, 7 hours, 8 hours, etc.). For example, maintaining may mean maintaining the temperature of the thermal regulator region at the phase temperature for at least 1 hr. In some variations, maintaining comprises maintaining the temperature of the thermal regulator region at the phase temperature for at least 6 hrs.

In general, positioning may mean adjusting the thermal transfer region of the applicator to conform to the subject's head.

A method of enhancing sleep in a subject may include: positioning an applicator having a thermal regulator region comprising a plurality of bodies each enclosing a phase change material having a phase transition between about 10 degrees C. and about 40 degrees C. and a thermal transfer region in thermal communication with the thermal regulator region so that the thermal transfer region contacts the subject's forehead but does not contact the periorbital, cheek, top or back regions of the subject's head; and maintaining the temperature of the thermal transfer region at the phase transition temperature for more than about 30 minutes to enhance the subject's sleep.

### BRIEF DESCRIPTION OF THE DRAWINGS

FIG. 1 is a front view of one variations of a non-invasive brain thermal regulation applicator on the head of a subject.

FIG. 2 is a side view of the embodiment of the non-invasive brain thermal regulation applicator similar to the applicator shown in FIG. 1.

FIG. 3A is a front view of a variation of a non-invasive brain thermal regulation applicator on the head of a subject.

FIG. 3B is a side view of the non-invasive brain thermal regulation applicator of FIG. 3A on the head of a subject.

FIG. 4 is a cross-sectional view through one variation of a thermal regulation applicator.

FIG. 5 is a cross-sectional view through another variation of a thermal regulation applicator.

FIG. 6 is a cross-sectional view through another variation of a thermal regulation applicator.

FIG. 7 is a cross-sectional view through another variation of a thermal regulation applicator.

### DETAILED DESCRIPTION

In general, described herein are thermal regulation applicators that are specifically configured to be comfortably worn on the subject's head, to thermally regulate (e.g., hold to a predetermined temperature) specific regions of the subject's brain (e.g., the frontal cortex region/prefrontal cortex) while remaining comfortable, and sustaining the temperature of the specific region of the head at a desired temperature for a specific one or more periods of time. In general, these devices may include a thermal transfer region to be worn directly against the subject's skin (in the head region above the frontal cortex) and a thermal regulator region passively holding the predetermined temperature (or predetermined temperature range) which is in thermal contact with the thermal transfer region. All of the apparatuses (devices and systems) herein described are intended to address the subjects comfort while the applicator is maintained in a position above the pre-frontal (or frontal) cortex.

The thermal transfer region may be temperature regulated by any appropriate mechanism, particularly passive thermal regulator regions. In some variations, the thermal transfer region may be thermally regulated by a phase change material forming the thermal regulator region. There are many types of phase change materials that could be utilized. A phase change material (PCM) is a substance with a high heat of fusion which, melting and solidifying at a certain temperature, is capable of storing and releasing relatively large amounts of energy. Heat is absorbed or released when the material changes from solid to liquid and vice versa; thus, PCMs are classified as latent heat storage (LHS) units.

PCMs latent heat storage can be achieved through solid-solid, solid-liquid, solid-gas and liquid-gas phase change. However, the phase change used for PCMs is typically the solid-liquid change, as liquid-gas phase changes are not typically practical for use as thermal storage due to the large volumes or high pressures required to store the materials when in their gas phase. Liquid-gas transitions do have a higher heat of transformation than solid-liquid transitions. Solid-solid phase changes are typically very slow and have a rather low heat of transformation.

Solid-liquid PCMs typically behave like sensible heat storage (SHS) materials; their temperature rises as they absorb heat. Unlike conventional SHS, however, when PCMs reach the temperature at which they change phase (their melting temperature) they absorb large amounts of heat at an almost constant temperature. The PCM continues to absorb heat without a significant rise in temperature until all the material is transformed to the liquid phase. When the ambient temperature around a liquid material falls, the PCM solidifies, releasing its stored latent heat. A large number of PCMs are commercially available in any required temperature range from −5 up to 190° C. Within the human comfort range of 20° to 30° C. (or within 10 degrees C. to 40 degrees C., or within 14 degrees C. to 40 degrees C., etc.), some PCMs are very effective. They store 5 to 14 times more heat per unit volume than conventional storage materials such as water, masonry or rock.

The brain temperature-regulating applicator apparatuses described herein (which may be referred to as non-invasive frontal or pre-frontal cortical stimulation regions) may use one or more phase change materials to thermally regulate a region of the subject's head, and therefore a region of the subject's underlying cortex (pre-frontal/frontal cortex) within the therapeutic range to enhance sleep and/or sleep onset. However, such devices should also be configured so that they can be comfortably worn. For example, they must conform to the subject's head over the appropriate region, and must be sufficiently light and compact (and in some variation flexible) so that they do not disrupt or prevent sleep, and must prevent tangling and/or disturbing the subject wearing the device while sleeping, including moving while sleeping.

Typically, phase change materials that change from a solid to either a liquid or gas exhibit limited conformability when in the solid state. This lack of conformability impacts the subjects overall comfort. Phase change materials are used in many applications to include relief from pain, swelling and stress reduction, however such materials have not previously been described as part of a device that is capable of enhancing sleep, including reducing sleep onset.

Any of the applicators described herein may be configured to applying cooling to enhance sleep, as demonstrated, for example, in U.S. Pat. No. 8,236,038, to the frontal cortex to enhance sleep. Thus, and of the applicators described herein may be configured to cool the subject's head over the pre-frontal/frontal cortex to a temperature that is between about 0 degrees C. and about 35 degrees C. (e.g., a temperature between about 10 degrees C. and 30 degrees C., a temperature between about 14 degrees C. and 30 degrees C., etc.) The temperature may be selected from within this range and held relatively constant at that temperature for some predetermined amount of time.

Any of the applicators described herein may also (or alternatively) be configured to apply generally “warming” (warming relative to the surface temperature of the subject) to the patient's head, e.g., between about 30 degrees C. and about 40 degrees C., e.g., between about 32 degrees C. and about 38 degrees C., etc. Warming has surprisingly been shown recently to enhance sleep in some patients; and particularly warming provide specifically (and/or exclusively) over the pre-frontal/frontal cortical region (e.g., forehead, etc.), and sustained at a relatively constant temperature for a predetermined period of time (e.g., 15 min, 30 min, 1 hour, 2 hours, 3 hours, 4 hours, 5 hours, etc.). As described in greater detail below, for variations in which the applicator includes a phase change material as part of the thermal regulator, the phase change material may be chosen so that appropriate warming/cooling temperature is select. Further, before operation of the applicator, the applicator (or at least the thermal regulator portion of the applicator including the phase change material) may be cooled or heated beyond the phase transition temperature so that the applicator will passively remove or apply thermal energy once applied to the subject.

In some variations of the apparatuses and systems described herein, the phase change material may be formulated to target a specific temperature for the phase change to occur that would be most beneficial for enhancing sleep, such as sleep quality, onset, duration, and/or for treating insomnia. The specific temperature may be a temperature that is not perceived as uncomfortably cold when cooling temperatures are applied (e.g., typically greater than or about 10° C., e.g., about 14° C.). In warming variations, other specific temperatures could be targeted between about, for example, 36° C. to about 44° C. (e.g., 38° C., 40° C., etc.) The thermal capacity of the phase change material would be sufficient to maintain the targeted temperature for a time period ranging from about 15 minutes to over 480 minutes (e.g., over about 15 min., over about 30 min., over about 45 min, over about 1 hr., over about 6 hrs., etc.)

The phase change material may be encapsulated in a bio-compatible material suitable for extended contact with the subject's skin, or for contact with a thermal transfer region that directly contacts the skin. The encapsulating material may be flexible and may act as a thermal conductor. Encapsulated phase change material could be used in conjunction with a mold to form the material into the shape of the forehead while being cooled below the phase change temperature/heated above the phase change temperature (e.g., if the applicator is intended to apply cooling). The shape of the mold could be generic based upon standard anatomy measurements, or could be custom shaped to the subjects head above the prefrontal cortex.

In some variations, the phase change material may be encapsulated into one compartment within the applicator. For example, a phase change material may be present in a large compartment

In some variations, the phase change material is formulated to maintain a high level of flexibility to enhance conformability and comfort.

In some variations, the phase change material is encapsulated into many compartments within the applicator allowing the applicator to become form fitting over the prefrontal cortex. The size of the compartments may be the same or may vary by location over the pre frontal cortex. In some variations, the compartments could be connected or independent to each other.

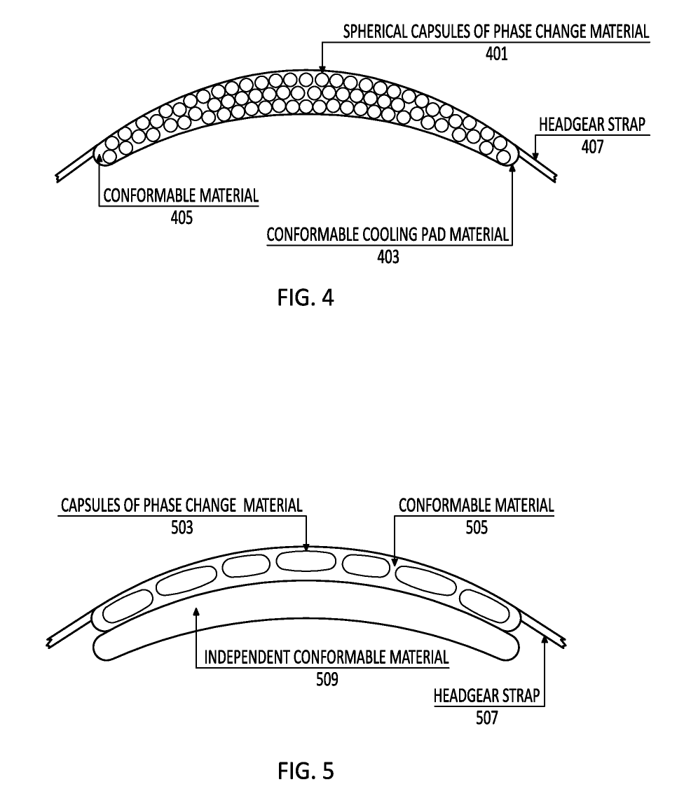

In some variations, the phase change material may be encapsulated into individual capsules or containers within the applicator, as shown in FIGS. 4 and 5. The capsules 401 may have a uniform size and shape or the size and shape could be varied to achieve enhanced conformability. Additionally, the size and shape of the capsules could be varied to develop specific thermal characteristics across the applicator. In some variations, different phase change materials with different solidification temperatures may be used to provide a matrix of regions or capsules in order to provide a broader range of temperatures. In such an application, one phase change material solidification temperature could be used as the preferred applicator temperature with one or more other phase change materials used to maintain a second or third temperature sequentially. In FIG. 4, the applicator is shown in cross-section from a top view. The applicator may include a headgear, e.g., a strap 407, for holding the applicator to the subject's head over the appropriate region. The thermal regulation region 401 including the phase-change material maybe in thermal communication with a thermal transfer material 405 for transferring thermal energy between the thermal regulator bodies (capsules 401) and the surface of the applicator that contacts the subject and includes a thermal transfer region 403. In FIG. 4, the thermal transfer region is configured as a pad. The applicator is generally configured so that other region of the applicator do not conduct the thermal energy to/from the thermal regulator bodies and the patient's head in regions that are not over the subject's frontal (prefrontal) cortex. For example, the subject's eyes, cheeks, back of the head, etc.

FIG. 5 illustrates another example of an applicator including a phase change material forming a passive thermal regulator. The thermal regulator is generally conformable, as the phase change material 503 is encapsulated/held in bodies within the thermal regulator, and the body of the thermal regulator is conformable; the body 505 may be formed of a material including a fabric having a relatively high thermal conductivity. The applicator may also include a thermal transfer region 509 that transmits thermal energy to the subject's head and is conformable so that it can be comfortably worn. The thermal transfer region may also be disposable/replaceable. In any of these variations the thermal transfer region may be breathable or configured so that it will absorb sweat and/or allow some air exchange (e.g., may include pores, etc.) The applicator may also include a headgear (e.g., strap 507) to hold the device to the subject's head.

In some variations, the phase change material could be mixed with other materials to form a matrix of materials such that the phase change material in the solid form would be suspended within other materials. Such a matrix may produce a more flexible and comfortable applicator.

As mentioned, in some variations, the phase change material could be attached to an interface material. The interface material may provide a higher degree of formability to the subject's anatomy above the pre frontal cortex than could be achieved by the phase change material encapsulations previously discussed. The interface material is typically a thermal transfer region having a relatively high thermal conductivity. For example, a thermal transfer region could be a gel material with high thermal conductivity, water or other formable materials that would enhance subject comfort, as illustrated in FIG. 7. In FIG. 7, the phase change material is a conformable phase change material 705, which is in thermal communication with a thermal transfer region 703 that is also conformable. The applicator may include a headgear (such as a strap 707).

Examples of phase change material formulations that could be used for sleep enhancement include inorganic (e.g., salt hydrates), eutectics (Organic-organic, organic-inorganic, inorganic-inorganic compounds, including paraffins), and Hygroscopic materials.

In use, any of the phase change material devices described herein may be prepared by cooling or warming prior to application. For applicators intended to cool the subject's frontal/prefrontal cortex, prior to placing the applicator in position on the subject's forehead over the brain (e.g., frontal, prefrontal) region of interest, the applicator may be cooled to, or in some variations, below, the phase change temperature by any appropriate method. In some variations the applicator is cooled by placing in a refrigerator until the phase change occurs. In other variations, a bedside cooling device is used to achieve the required temperature for phase change to occur. This bedside cooling device could cool the applicator utilizing any readily available refrigeration techniques including, but not limited to: compressor driven and/or Pelletier refrigeration. In any of the system/device variations described, a cooling mold could be used to pre-shape the applicator to conform to the subject's forehead. This may provide for additional comfort and may enhance the thermal contact of the applicator. Once positioned on the subject's forehead the applicator would maintain the temperature in a narrow temperature range due to the heat absorbing characteristics of the phase change material. Similarly, in variations in which the applicator is intended to warm the subject by passively holding the thermal regulator at a warming temperature (above ambient skin temperature), the applicator or just the thermal regulator region could be heated above the transition temperature of the phase change material in the applicator. For example, a phase change material could be a Sodium Acetate solution that produces heat when it crystalizes. The crystallization of Sodium Acetate occurs when heterogeneous nucleation is initiated (e.g., a nucleation agent that is below the phase transition temperature).

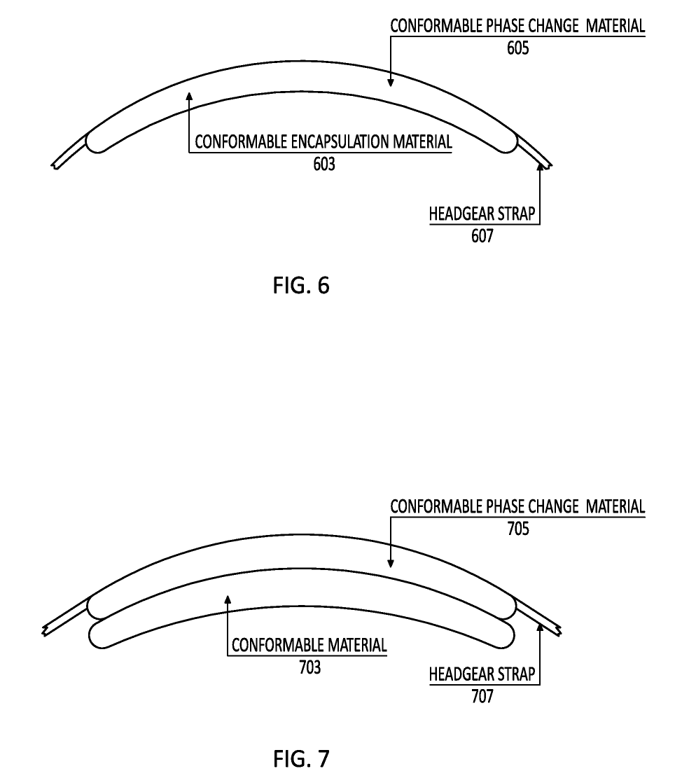

FIG. 6 shows a variation of an applicator in which the phase change material 605 his generally conformable, and is encapsulate in a material 603 having a sufficiently high thermal conductivity (at least on the side of the applicator facing the subject) to form a thermal transfer region. In some variations an additional thermal transfer region (not shown) may be used. The applicator may also include a headgear 607 for holding the applicator against the subject's head in the appropriate position, so that the thermal transfer region is adjacent to the forehead and other regions above the frontal/prefrontal cortex.

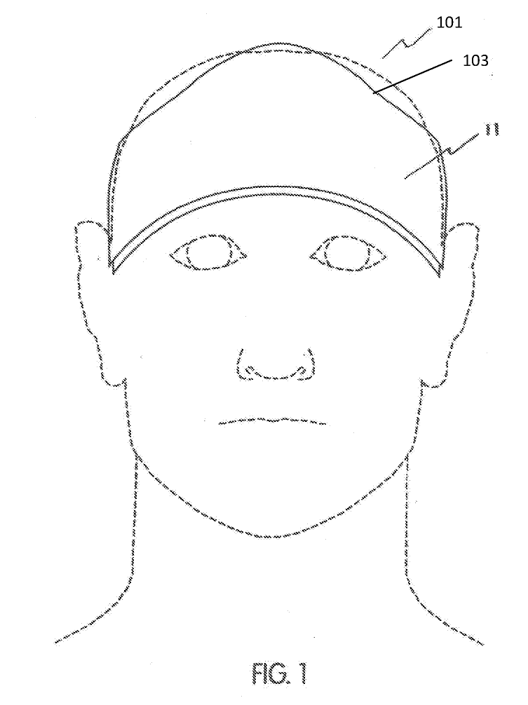

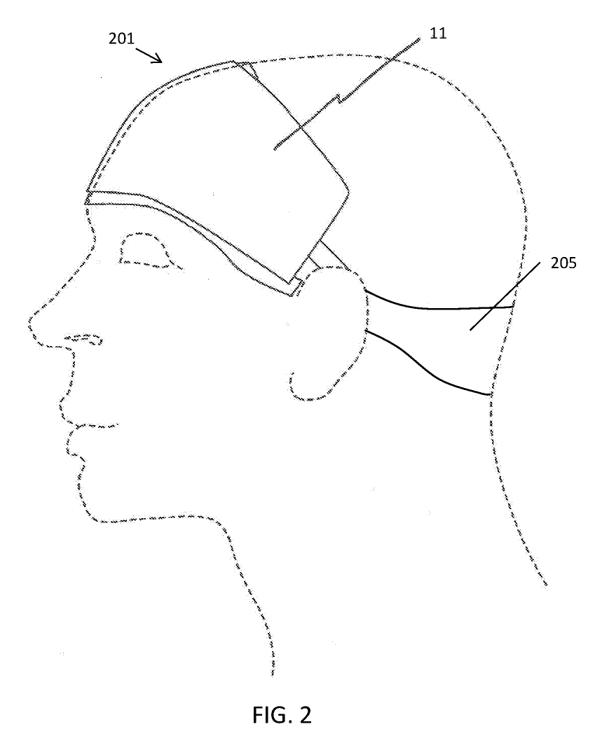

FIGS. 1 and 2 illustrate one variation of an applicator being worn by a subject 101. In this variation the applicator includes an outer shell 103 over a thermal regulator 11 that is positioned above the frontal cortex. The shell may form a headgear holding the device to the subject's head. The applicator may also include a thermal transfer region (not visible in FIG. 1) on the inner surface just between the thermal regulator 11 portion within the applicator and the subject's forehead/scalp, over the frontal/prefrontal cortex.

Two variations of side views are shown in FIG. 2. In FIG. 2, the applicator 201 housing the thermal regulator 11 includes a headgear comprising the outer shell and a strap 205 that goes around the back of the subject's head. The strap 205 is configured to be comfortably worn during sleep. The strap does not transfer thermal energy between the thermal regulator 11 and the other portions of the subject's head.

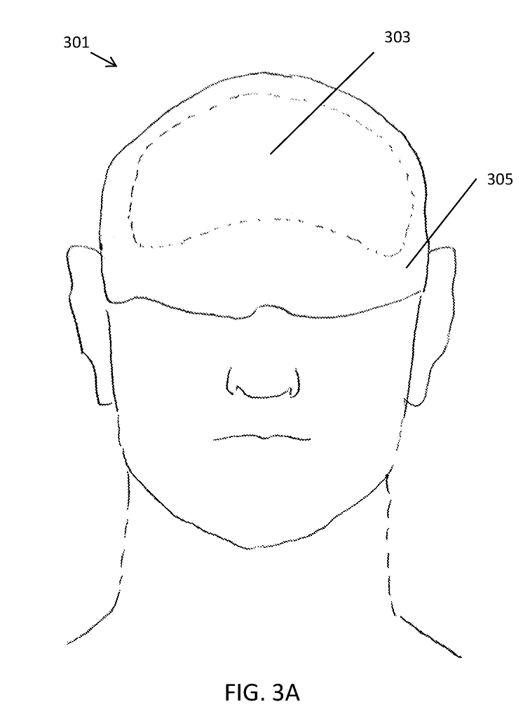

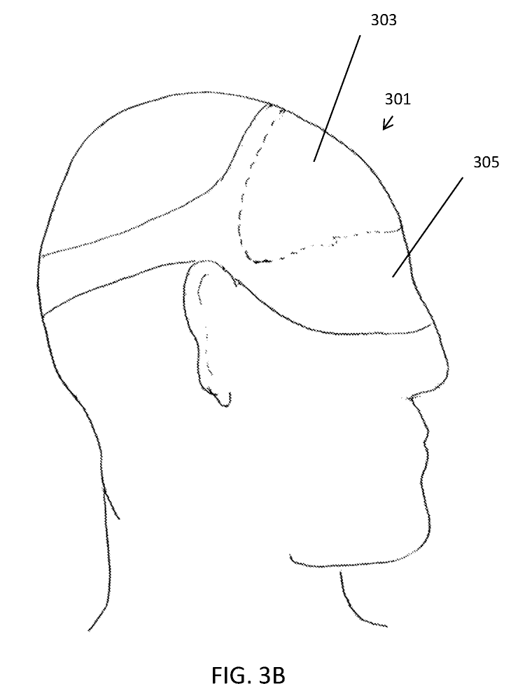

FIG. 3A-3B illustrate another variation of an applicator that can be used as described herein. In this variation, the applicator includes an internal thermal regulator 303 that is surrounded on the outside surfaces by a headgear 305 including a region covering the subject's eyes. An internal thermal transfer region (not visible in FIG. 3A) is present between the thermal regulator and the region of the applicator that is worn over the subject's prefrontal cortex. Other regions of the applicator (e.g., the headgear), including the region over the subject's eyes, is not a thermal transfer region, and does not transfer thermal energy between the thermal regulator and that portion of the subject's head. FIG. 3B shows a side view of the applicator of FIG. 3A worn on a patient.

### Evaporative Cooling

In some variations the apparatuses and systems could utilize evaporative cooling to maintain the temperature of the applicator at the desired therapeutic temperature to enhance sleep. There are many forms of evaporative cooling commercially available, however, to date no effective evaporative cooling systems or devices have been formulated or configured specifically for sleep enhancement or the treatment of insomnia. As described herein, any appropriate evaporative cooling systems, devices, or materials could be engineered to meet the specific requirements for sleep enhancement and the treatment of insomnia.

For example, in some variations, the evaporative cooling device includes sodium polyacrylate crystals to enhance moisture retention. In some variations the evaporative cooling applicator is manufactured from hydrophilic fibers specifically formulated and produced in a manner to enhance moisture retention. In such a variation the applicator would be produced from the hydrophilic textile material and would be shaped to conform to cover the subject's forehead or possibly the entire frontal cortex area by common textile manufacturing techniques. The hydrophilic textile material would be held in place by a headgear or adjustable strap. In use, the hydrophilic material would be saturated in water prior to being placed in position on the subject and cooling of the pre frontal cortex area would occur from the evaporation of the moisture contained within the applicator. FIGS. 1 &2 indicate the relative shape and location of the evaporative material applicator on a subject. The type and quantity of the selected evaporative material used would ensure sufficient cooling for at least 15 minutes. In some variations, the evaporative cooling material is shaped to form an applicator comprising a thermal transfer region in communication with the subject's skin over the prefrontal cortex.

An evaporative cooling device may be configured so that the applicator does not leak or spill water. For example, the evaporative cooling device maybe sealed around all but one or more evaporative air ports; the air ports may be configured to provided fluid locks that minimize or prevent fluid leakage.

### Headgear

As discussed above, any of the applicators described herein may be used with a headgear that is specifically configured to maintain the applicator in thermal contact with the subject's head in a snug but non-restrictive manner. Thus, in some variations the applicator is held in position with a headgear. The headgear maintains thermal contact of the applicator to provide regional cooling of the area in proximity to the frontal cortex. The headgear may be configured to allow the subject to adjust the amount of contact pressure applied to the applicator and to adjust for comfort, while maintaining the position of the thermal transfer region of the applicator over the frontal cortex. The headgear can be configured from a variety of materials. In some variations where the cooling is achieved by a phase change material the headgear could include an insulation material that covers the surface of the applicator distal to the subject to reduce parasitic heat from accelerating the phase change. The insulation material may be an elastic material or covered with an elastic material that would induce increased contact pressure of the applicator to the subject's forehead when stretched by adjustable straps wrapping the circumference of the subjects head. The adjustable straps can be produced from any suitable material either exhibiting an elastic characteristic or not and incorporate any adjustable feature readily available such as Velcro, snaps, buttons, hooks etc. In some variations, the adjustment features may allow for macro adjustment of the circumferential head size and secondary adjustment features to micro adjust specific areas of the applicator to ensure optimal thermal contact and comfort. In some variations, the headgear is produced from an elastic material in fixed sizes without adjustability i.e. small, medium and large.

The headgear utilized for an evaporative cooling applicator may be configures so that it does not cover the distal side of the evaporative cooling material with an insulation layer as this would inhibit the evaporative process.

The headgear may be reusable and/or separate from the applicator. Alternatively the headgear may be integral with the applicator. The headgear may be constructed of a singular piece to allow thermal contact for the regional cooling. For example, the headgear may include a thermal transfer region oriented so that it is positioned against the head over the subject's frontal (and/or prefrontal) cortex region. The other regions of the headgear may be thermally insulated. In general, the headgear may include a pocket or clips to secure an applicator against the subject's head. In some variations the applicator is one or more standard sizes, and the headgear is provided in different sizes that may fit the standard size(s) of the applicator. The headgear is typically adjustable. In general the headgear may be cushioned, particularly in the regions surrounding the applicator.

In some variations, the headgear may be constructed of multiple pieces for better thermal contact and comfort of the patient.

As mentioned, in some variations, the headgear may be constructed to allow adjustment to allow for better thermal contact and comfort of the patient.

As mentioned, a headgear may be for single use, or it may be reusable. For example, in some variations, the headgear may be a singular use and replaced on each application.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An applicator to enhance sleep by regulating the temperature of a subject's frontal cortex when worn, the applicator comprising:
a removable thermal regulator region comprising a phase change material configured to cool; and
a headgear configured to hold the removable thermal regulator region, the headgear including:
a thermal transfer region in thermal communication with the thermal regulator region when the removable thermal regulator region is held by the headgear, wherein the thermal transfer regions is configured to conform to and to contact a subject's forehead so that the thermal transfer region is positioned against the subject's head over the frontal cortex so that the phase change material may cool the frontal cortex; and
a strap configured to hold the applicator against the subject's head when the subject is sleeping.

2. The applicator of claim 1, wherein the phase change material has a phase transition between about 10 degrees C. and about 30 degrees C.

3. The applicator of claim 1, wherein the phase change material has a phase transition between about 10 degrees C. and about 18 degrees C.

4. The applicator of claim 1, wherein the headgear is configured to thermally insulate the removable thermal regulator other than the thermal transfer region.

5. The applicator of claim 1, wherein the thermal regulator comprises a plurality of capsules, wherein each capsule encapsulates the phase change material.

6. The applicator of claim 1, wherein the thermal regulator region comprises a plurality of capsules each encapsulating the phase change material, wherein the capsules are arranged in a matrix of thermally conductive and conformable material.

7. The applicator of claim 1, wherein the phase change material comprises a mixture of two or more different phase change materials.

8. The applicator of claim 1, wherein the thermal regulator is configured so that the phase change material is maintained at about the phase transition temperature for greater than about 30 minutes when the applicator is worn by a subject.

9. The applicator of claim 1, wherein the thermal regulator is configured so that the phase change material is maintained at about the phase transition temperature for greater than about 6 hours when the applicator is worn by a subject.

10. The applicator of claim 1, wherein the thermal transfer regions comprises a material having a thermal conductivity of greater than about 0.1 watts per meter kelvin (W/(m*K)).

11. The applicator of claim 1, wherein the thermal transfer region is configured to position the thermal regulator over just the frontal cortex and immediately adjacent regions.

12. The applicator of claim 1, wherein the thermal transfer region is configured to contact the subject's forehead but not to contact the subject's periorbital or cheek regions of the subject's face when the applicator is worn by the subject.

13. The applicator of claim 1, wherein the thermal transfer region is configured to contact the subject's forehead but not to contact the back of the subject's head when the applicator is worn by the subject.

14. The applicator of claim 1, wherein the thermal transfer region comprises a layer of thermally conductive material configured to contact the subject's forehead when the applicator is worn by the subject.

15. The applicator of claim 1, wherein the strap is configured as a headgear.

16. An applicator to enhance a subject's sleep by regulating the temperature of the frontal cortex when worn, the applicator comprising:
a removable thermal regulator region enclosing a phase change material having a phase transition between about 10 degrees C. and about 30 degrees C.; and
a headgear configured to hold the removable thermal regulator region, the headgear including:
a thermal transfer region in thermal communication with the thermal regulator region when the removable thermal regulator region is held by the headgear, wherein the thermal transfer regions is configured to conform to and to contact a subject's head over the frontal cortex,
further wherein the thermal transfer region is configured to contact and the subject's forehead but not to contact the subject's periorbital or cheek regions of the subject's face to regulate temperature when the applicator is worn by the subject, wherein the headgear is configured to thermally insulate the removable thermal regulator other than the thermal transfer region; and
a strap configured to hold the applicator against the subject's head when the subject is sleeping.

17. The applicator of claim 16, wherein the thermal regulator is configured so that the phase change material is maintained at about the phase transition temperature for greater than about 30 minutes when the applicator is worn by a subject.

18. The applicator of claim 16, wherein the thermal transfer region is configured to contact the subject's forehead but not to contact the back of the subject's head when the applicator is worn by the subject.

19. The applicator of claim 16, wherein the thermal transfer region comprises a layer of thermally conductive material configured to contact the subject's forehead when the applicator is worn by the subject.

20. The applicator of claim 16, wherein the strap is configured as a headgear.

21. A method of enhancing sleep in a subject, the method comprising:
combining a removable thermal regulator region with a headgear;
positioning the headgear holding the removable thermal regulator region, wherein the removable thermal regulator region comprises a phase change material in thermal communication with a thermal regulator region of the headgear so that the thermal transfer region contacts the subject's forehead but does not contact the periorbital or cheek regions of the subject's face; and
maintaining a temperature of the thermal transfer region at between 10 degrees C. to 30 degrees C. to enhance the subject's sleep.

22. The method of claim 21, wherein positioning comprises positioning the applicator so that the thermal transfer region does not contact the top or back of the subject's head.

23. The method of claim 21, wherein positioning comprises adjusting a headgear to hold the applicator to the subject's head.

24. The method of claim 21, wherein maintaining comprises maintaining the temperature of the thermal regulator region at the phase transition temperature for at least 30 minutes.

25. The method of claim 21, wherein maintaining comprises maintaining the temperature of the thermal regulator region at the phase temperature for at least 1 hr.

26. The method of claim 21, wherein maintaining comprises maintaining the temperature of the thermal regulator region at the phase temperature for at least 6 hrs.

27. The method of claim 21, wherein positioning comprises adjusting the thermal transfer region of the applicator to conform to the subject's head.

28. A method of enhancing sleep in a subject, the method comprising:
combining a removable thermal regulator region with a headgear;
positioning the headgear holding the removable thermal regulator region, wherein the removable thermal regulator region comprises a phase change material in thermal communication with a thermal regulator region of the headgear so that the thermal transfer region contacts the subject's forehead but does not contact the periorbital, cheek, top or back regions of the subject's head; and
maintaining the temperature of the thermal transfer region at between about 10 degrees C. and about 30 degrees C. for more than about 30 minutes to enhance the subject's sleep.
